(19) United States
(12) Patent Application Publication
(10) Pub. No.: US 2008/0199501 A1
(43) Pub. Date: Aug. 21, 2008

(54) ARTICLE/COMPOSITION COMBINATION

(75) Inventor: Anthony POTIN, Montrouge (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 12/031,882

(22) Filed: Feb. 15, 2008

Related U.S. Application Data
(60) Provisional application No. 60/892,270, filed on Mar. 1, 2007.

(30) Foreign Application Priority Data
Feb. 21, 2007 (FR) 07 53397

(51) Int. Cl. A61K 9/00; A61Q 5/00

(52) U.S. Cl. 424/401; 424/400; 424/402

(57) ABSTRACT

The invention relates to a cosmetic article having a water-insoluble substrate and a composition added to or impregnated onto the substrate containing at least one aqueous phase, at least one oily phase and one or more semi-crystalline polymers. This article can constitute in particular a wipe which can be used especially for caring for the skin and/or hair and/or for cleaning and/or removing make-up from the skin.

### REFERENCE TO PRIOR APPLICATIONS

This application claims priority to U.S. provisional application 60/892,270 filed Mar. 1, 2007, and to French patent application 0753397 filed Feb. 21, 2007, both incorporated herein by reference.

### FIELD OF THE INVENTION

The invention relates to an article, more particularly a cosmetic article such as a wipe, comprising at least one water-insoluble substrate and a composition comprising at least one semi-crystalline polymer, and to the uses of the article in, e.g., the cosmetic and dermatological fields, in particular for caring for and treating the human skin, cleaning and/or removing make-up from human skin, most especially the face, and caring for the hair.

Additional aspects and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Cosmetic wipes are generally composed of a substrate made of a material of natural or synthetic origin, which is preferably a non-woven material but which can also be a foam or woven material, the substrate being impregnated with a composition suitable for the desired objective, for example cleaning or removing make-up from the skin or eyes or also caring for the skin or hair. Such wipes are commonly used as they are valued for their practical side owing to the fact that they are disposable and that they are impregnated with the necessary and sufficient amount of treating or cleaning product. The use of these wipes avoids the handling and transportation of bottles comprising cosmetic compositions.

The impregnation of the substrates by the impregnating composition can be carried out according to different techniques, such as spraying or quenching. However, these techniques can be used only if the impregnating compositions are sufficiently fluid and have a viscosity close to that of water. This is because it is not possible to correctly wet the substrate when the compositions are too viscous as the substrate is then incorrectly impregnated and, furthermore, it is difficult to cut, fold and pack into bags. In addition, the article obtained is unpleasant to use as the impregnating product remains at the surface of the substrate and does not homogeneously impregnate it, with the result that some regions of the article comprise too much product while others are without it or comprise too little of it.

Furthermore, the fluid compositions which impregnate the wipes exhibit the disadvantage of comprising little oil and, for this reason, of having a care effect on the skin or a make-up-removing power which are regarded as inadequate. Furthermore, the wipes obtained exhibit a lack of comfort on application.

There exist wipes comprising a composition based on oils which are used for caring for the skin (impregnation with body oils) or for removing make-up from the skin (impregnation with make-up-removing oils) but, during application to the skin, these wipes leave a greasy film which the wipe, itself impregnated with oil, cannot remove. This is particularly unpleasant with regard to the eye when the wipe is used for removing make-up from the eyes. Furthermore, wipes comprising oils in combination with surfactants, which are used after moistening with a small amount of water, the addition of water making it possible to obtain an emulsion by dispersion of the mixture of oil and of surfactant in the water, but the emulsion thus obtained remains very liquid, without consistency, and has a tendency to flow onto the substrate, which is unpleasant for the user.

Thus, the compositions for impregnating wipes used to date are either too greasy or lack consistency and do not make it possible to obtain a creamy texture, which are important criteria for the achievement of good comfort in use, both in caring for and in removing the make-up from the skin. In point of fact, the comfort contributed by a cosmetic product, during the use thereof and immediately after the use thereof, is just as important as its effectiveness. The fact of obtaining a cream and not a fluid contributes a comfort to the skin which is highly valued by the user. Furthermore, the fact of having a thicker composition which is richer in oils also makes it possible to invert the effectiveness of the product in caring for and/or removing make-up from the skin.

### SUMMARY OF THE INVENTION

Thus, the need remains to have available an article (wipe or compress or foam, etc.) which can give, during application of the skin, a smooth composition, such as a cream, this article being simultaneously easily used, comfortable on application and effective.

The inventor has found, surprisingly, that a composition in the form of an emulsion, comprising one or more semi-crystalline polymers, can be impregnated, etc., homogeneously by conventional means onto a wipe or any other water-insoluble substrate. The composition according to the invention exhibits the advantage of giving very homogeneous impregnation onto the wipe, which is not the case with other oil-phase thickeners.

### DETAILED DESCRIPTION OF THE PREFERRED EMBODIMENTS

One subject-matter of the present invention is thus an article, for example a cosmetic article, comprising (A) a water-insoluble substrate, comprising one or more layers, and (B) a composition added to or impregnated onto the substrate, comprising at least one aqueous phase, at least one oily phase, and one or more semi-crystalline polymers.

The term “cosmetic article” is understood here to mean the combination composed of a water-insoluble support and a cosmetic composition added to or impregnated onto the support. This article can in particular be a wipe but it can also have any appropriate form as described below. The water-insoluble substrate must be absorbent and sufficiently strong not to disintegrate during the use thereof.

The composition of the invention preferably has the consistency of a cream and preferably exhibits a viscosity of preferably from 1 Pa·s to 15 Pa·s, more preferably of 1 to 10 Pa·s and better still from 1.5 Pa·s to 10 Pa·s, this viscosity being measured at ambient temperature (approximately 25° C.) with a Rheomat RM 180 device, spindles 1 to 4 depending on the viscosity of the composition.

The article according to the invention is preferably moist to the touch. It preferably exhibits the advantage of being comfortable during application to the skin and having a nourishing effect due to the presence of an oily phase. Moreover, impregnation is very homogeneous by virtue of the presence of the semi-crystalline polymers.

In addition, this article is very easy to handle during use. When it is an article for caring for the skin, it preferably is applied to the skin so as to remain bonded to the skin, on which it should remain for a time sufficient for the composition to impregnate the skin, it being possible for this time to range, for example, from 5 to 30 minutes. The article is then removed and the excess composition present on the skin is wiped off. This article can also constitute a care mask. It can also be used in the same way on the hair.

When it is used for cleaning or removing make-up from the skin, it is passed over the skin, while possibly leaving it applied for a time sufficient for the make-up products to be dissolved in the impregnating composition of the article, and then the skin is wiped. The skin can also optionally be rinsed subsequently.

The article according to the invention is in particular a cosmetic article appropriate for caring for and/or treating the skin of the face, body or hands and for cleaning or removing make-up from the skin of the face and/or body. It can also be used for caring for the hair and for removing make-up from the eyes.

The article according to the invention can have any form appropriate to the desired objective. It can constitute a wipe but it can also be in the form of a glove, of a mitten or in any other form appropriate for particle use on the face or the body, for example in the form of a face with holes for the sites of the eyes, nose and/or mouth, or in the form of a make-up-removing fingerstall for application in removing make-up from the eyelashes, or in the form of a single- or double-sided disc which can in particular comprise two sides impregnated with different compositions. The glove form is particularly suitable for the treatment of dry or chapped hands as the hand can be left in the glove for a time sufficient for the composition to impregnate the skin of the hands and to contribute to it the beneficial effects of oils present therein. The article can also comprise a rough surface which makes possible the exfoliation (scrubbing) of the skin.

Another subject-matter of the invention is the use of the article as defined above for caring for the skin or hair and/or cleaning and/or removing make-up from and/or scrubbing the skin.

The composition used according to the invention for impregnating the water-insoluble substrate, as it is preferably intended for topical application, preferably comprises a physiologically acceptable medium, that is to say the medium compatible with the skin, mucous membranes, hair and scalp.

The composition of the invention comprises at least one aqueous phase and at least one oily phase and it can be in the form of a water-in-oil (W/O) emulsion or of an oil-in-water (O/W) emulsion or of a multiple (W/O/W or O/W/O) emulsion. Preferably, the composition is in the form of a W/O emulsion.

Semi-Crystalline Polymers

The composition according to the invention comprises at least one semi-crystalline polymer, preferably one derived from acrylic or methacrylic acid. This polymer is generally in the oily phase. The term “semi-crystalline polymer” is understood to mean, within the meaning of the invention, polymers comprising a crystallizable part, pendent chain or block in the backbone, and an amorphous part in the backbone, and exhibiting a first-order reversible phase-change temperature, in particular a melting point (solid-liquid transition). When the crystallizable part is a block of the polymer backbone, this crystallizable block is of different chemical nature from that of the amorphous blocks; the semi-crystalline polymer is, in this case, a sequential polymer, for example of the diblock, triblock or multiblock type.

Advantageously, the semi-crystalline polymer or polymers of the composition of the invention have a number-average molecular weight M̅n of greater than or equal to 2000, for example ranging from 2000 to 800 000, preferably from 3000 to 500 000, for example from 4000 to 150 000 and better still from 4000 to 99 000.

In the composition according to the invention, the semi-crystalline polymers are advantageously soluble in the oily phase to at least 1% by weight, at a temperature greater than their melting point. Apart from the crystallizable chains or blocks, the blocks of the polymers are amorphous. The term “crystallizable chain or block” is understood to mean, within the meaning of the invention, a chain or block which, if it were alone, would reversibly change from the amorphous state to the crystalline state depending upon whether the temperature is above or below the melting point. A chain within the meaning of the invention is a group of atoms which is in the pendent or side position with respect to the backbone of the polymer. A block is a group of atoms belonging to the backbone, which group constitutes one of the repeat units of the polymer.

Preferably, the polymeric backbone of the semi-crystalline polymers is soluble in the oily phase.

Preferably, the semi-crystalline polymers used in the composition of the invention exhibit a melting point, m.p., of less than 70° C. (25° C.≦m.p.<70° C.), this temperature being at least equal to the temperature of the keratinous substance which has to receive the composition according to the invention, in particular the skin. The melting point can be measured in particular by any known method and in particular with a differential scanning calorimeter (DSC).

Preferably, the crystallizable blocks or chains of the semi-crystalline polymers represent at least 30% of the total weight of the polymer and better still at least 40% of the total weight of the polymer. The semi-crystalline polymers comprising crystallizable blocks used according to the invention are preferably block or multiblock polymers. They can be obtained by polymerization of monomers comprising reactive double bonds (or ethylenic bonds) or by polycondensation. When the polymers of the invention are polymers comprising side chains which are crystallizable, they are advantageously in the statistical or random form.

The semi-crystalline polymers of the invention are generally of synthetic origin. They are preferably chosen from polymers (homopolymers or copolymers) carrying at least one crystallizable side chain and polymers (homopolymers or copolymers) carrying, in the backbone, at least one crystallizable block, such as those described in the document U.S. Pat. No. 5,156,911. The crystallizable side chain(s) or block(s) are hydrophobic.

According to a preferred embodiment of the invention, the semi-crystalline polymers are chosen in particular from homopolymers and copolymers resulting from the polymerization of at least one monomer comprising crystallizable chain(s), the crystallizable chain being chosen from alkyl chains comprising at least 11 carbon atoms and at most 40 carbon atoms and better still at most 24 carbon atoms. They are in particular alkyl chains comprising at least 12 carbon atoms and they are preferably alkyl chains comprising from 14 to 24 carbon atoms (C₁₄-C₂₄). They are hydrocarbon alkyl chains (carbon and hydrogen atoms) or fluorinated or perfluorinated alkyl chains (carbon atoms, fluorine atoms and optionally hydrogen atoms). When they are fluorinated or perfluorinated alkyl chains, they comprise at least 11 carbon atoms, at least 6 carbon atoms of which are fluorinated. Alkyl chains are preferably hydrogen chains.

The term “alkyl” is understood to mean, within the meaning of the invention, a saturated group (not comprising unsaturation).

According to a specific embodiment of the invention, the semi-crystalline polymer is chosen from polymers resulting from at least one monomer comprising a crystallizable chain chosen from C₁₄-C₂₄ alkyl(meth)acrylates, C₁₁-C₁₅ perfluoroalkyl(meth)acrylates, N—(C₁₄ to C₂₄ alkyl)(meth)acrylamides, with or without a fluorine atom, vinyl esters comprising C₁₄ to C₂₄ alkyl or perfluoroalkyl chains, vinyl ethers comprising C₁₄ to C₂₄ alkyl or perfluoroalkyl chains, C₁₄ to C₂₄ α-olefins, para-alkylstyrenes with a C₁₄ to C₂₄ alkyl group, and the copolymers of these monomers obtained by copolymerization of these monomers with a hydrophilic monomer, preferably other than methacrylic acid, such as, for example, N-vinylpyrrolidone, hydroxyethyl acrylate, hydroxyethyl methacrylate or acrylic acid. Such copolymers can, for example, be copolymers of C₁₄-C₂₄ alkyl acrylate, C₁₄-C₂₄ alkyl methacrylate, (C₁₄-C₂₄ alkyl)acrylamide or (C₁₄-C₂₄ alkyl)methacrylamide with N-vinylpyrrolidone, hydroxyethyl acrylate, hydroxyethyl methacrylate, acrylic acid, or their mixtures.

The semi-crystalline polymers of the composition of the invention can be non-crosslinked or partially crosslinked, as long as the degree of crosslinking does not interfere with their dissolution or dispersion in the oily phase by heating above their melting point. The crosslinking can then be chemically crosslinking, by reaction with a polyfunctional monomer during the polymerization. The crosslinking can also be physical crosslinking, which can then be due either to the establishment of dipolar or hydrogen-type bonds between groups carried by the polymer, such as, for example, dipolar interactions between carboxylate isomers, these interactions being in small amount and carried by the backbone of the polymer, or to phase aberration between the crystallizable blocks and the amorphous blocks carried by the polymer.

Preferably, the semi-crystalline polymers of the composition according to the invention are non-crosslinked.

According to a specific embodiment of the invention, the semi-crystalline polymer is chosen from homopolymers resulting from the polymerization of a monomer comprising a crystallizable chain chosen from saturated C₁₄-C₂₂ alkyl acrylates and saturated C₁₄-C₂₂ alkyl methacrylates and copolymers obtained by copolymerization of a monomer comprising a crystallizable chain chosen from saturated C₁₄-C₂₂ alkyl acrylates and saturated C₁₄-C₂₂ alkyl methacrylates with acrylic acid, in particular copolymers obtained by copolymerization of behenyl acrylates and acrylic acid or copolymers obtained by the copolymerization of stearyl acrylates and acrylic acid.

Mention may in particular be made, as homopolymers resulting from the polymerization of a monomer comprising a crystallizable chain chosen from saturated C₁₄-C₂₂ alkyl acrylates and saturated C₁₄-C₂₂ alkyl methacrylates, of those sold under the Intelimer® names by Landec, which are described in the booklet “Intelimer® Polymers”, Landec IP22 (Rev. 4-97). These polymers are in a solid form at ambient temperature. They carry crystallizable side chains and correspond to homopolymers of saturated C₁₄-C₂₄ alkyl acrylates or methacrylates. Mention may more particularly be made of stearyl acrylate homopolymer (Intelimer IPA-13.1) (INCI name: Poly C10-30 alkyl acrylate), behenyl acrylate homopolymer (Intelimer IPA-13.6) (INCI name: Poly C10-C30 alkyl acrylate), and their blends.

The amount of semi-crystalline polymer(s) in the composition of the invention can range, for example, from 0.1 to 20% by weight, preferably from 0.1 to 10% by weight and better still from 0.5 to 5% by weight, with respect to the total weight of the composition.

Oily Phase

The oily phase of the composition comprises one or more oils, in particular cosmetic oils. The amount of oily phase can range, for example, from 2 to 70% by weight, preferably from 5 to 50% by weight, better still from 10 to 40% by weight, with respect to the total weight of the composition.

The term “oil” is understood to mean a fatty substance which is liquid at ambient temperature (25° C.).

Mention may be made, as oils which can be used in the composition of the invention, for example, of:
hydrocarbon oils of vegetable origin, such as liquid triglycerides of fatty acids comprising from 4 to 10 carbon atoms, such as triglycerides of heptanoic acid or octanoic acid, or also, for example, sweet almond, sunflower, maize, soybean, cucumber, coriander, grape seed, sesame, hazelnut, apricot (Prunus armeniaca), macadamia, arara, castor or avocado oils, triglycerides of caprylic/capric acids, such as those sold by Stearineries Dubois or those sold under the names Miglyol 810, 812 and 818 by Dynamit Nobel, jojoba oil or shea butter oil;
synthetic esters and ethers, in particular of fatty acids, such as oils of formulae R¹COOR² and R¹OR² in which R¹ represents the residue of a fatty acid comprising from 8 to 29 carbon atoms and R² represents a branched or unbranched hydrocarbon chain comprising from 3 to 30 carbon atoms, such as, for example, purcellin oil or 660084 PCL-LIQUID from Symrise (mixture of cetearyl 2-ethylhexanoate and isopropyl myristate), isononyl isononanoate, isopropyl myristate, isopropyl palmitate, 2-ethylhexyl palmitate (or octyl palmitate), 2-octyldodecyl stearate, 2-octyldodecyl erucate or isostearyl isostearate; hydroxylated esters, such as isostearyl lactate, octyl hydroxystearate, octyldodecyl hydroxystearate, diisostearyl malate, triisocetyl citrate or heptanoates, octanoates or decanoates of fatty alcohols; polyol esters, such as propylene glycol dioctanoate, neopentyl glycol diheptanoate and diethylene glycol diisononanoate; and pentaerythritol esters, such as pentaerythrityl tetraisostearate;
linear or branched hydrocarbons of mineral or synthetic origin, such as volatile or non-volatile liquid paraffins and their derivatives, liquid petrolatum, polydecenes or hydrogenated polyisobutene, such as Parleam® oil;
fatty alcohols having from 8 to 26 carbon atoms, such as cetyl alcohol, stearyl alcohol and their mixture (cetearyl alcohol), octyldodecanol, 2-butyloctanol, 2-hexyldecanol, 2-undecylpentadecanol, oleyl alcohol or linoleyl alcohol;
fluorinated oils which partially comprise hydrocarbon and/or silicone, such as those described in the document JP-A-2-295912. Mention may also be made, as fluorinated oils, of perfluoromethylcyclopentane and perfluoro-1,3-dimethylcyclohexane, sold under the names of “Flutec PC1®” and “Flutec PC3®” by BNFL Fluorochemicals; perfluoro-1,2-dimethylcyclobutane; perfluoroalkanes, such as dodecafluoropentane and tetradecafluorohexane, sold under the names of “PF 5050®” and “PF 5060®” by 3M, or bromoperfluorooctyl, sold under the name of “Foralkyl®” by Atochem;
silicone oils, such as volatile or non-volatile polydimethylsiloxanes (PDMSs) comprising a linear or cyclic silicone chain which are liquid or pasty at ambient temperature, in particular cyclopolydimethylsiloxanes(cyclomethicones), such as cyclohexasiloxane; polydimethylsiloxanes comprising pendant alkyl, alkoxy or phenyl groups or alkyl, alkoxy or phenyl groups at the end of the silicone chain, which groups have from 2 to 24 carbon atoms; phenylated silicones, such as phenyl trimethicones, phenyl dimethicones, phenyl(trimethylsiloxy)diphenylsiloxanes, diphenyl dimethicones, diphenyl(methyldiphenyl)tri-siloxanes, (2-phenylethyl)trimethylsiloxysilicates and polymethylphenylsiloxanes;
and their mixtures.

The term “hydrocarbon oil” is understood to mean, in the list of the oils mentioned above, any oil comprising predominantly carbon and hydrogen atoms and optionally ester, ether, fluorinated, carboxylic acid and/or alcohol groups.

Emulsifiers

The composition can comprise one or more emulsifiers, the nature of which depends on the sense of the emulsion, which make possible the stabilization of the mixture of aqueous and oily phases. The amount (as active material) of emulsifier(s) preferably ranges from 0.01 to 10% by weight, better still from 0.05 to 5% by weight and even better still from 0.1 to 3% by weight of active material, with respect to the total weight of the composition.

These emulsifiers can be chosen from amphoteric, anionic, cationic or non-ionic emulsifiers, used alone or as a mixture. These are preferably non-ionic emulsifiers. These emulsifiers are appropriately chosen according to the continuous phase of the emulsion to be obtained (W/O or O/W). When the emulsion is a multiple emulsion, it generally comprises an emulsifier in the primary emulsion and an emulsifier in the external phase into which the primary emulsion is introduced.

Mention may be made, as emulsifiers which can be used for the preparation of the W/o emulsions, for example, of alkyl esters or alkyl ethers of sorbitan, of glycerol or of sugars; silicone surfactants, such as dimethicone copolyols, for example the mixture of cyclomethicone and dimethicone copolyol sold under the names DC5225C and DC3225C by Dow Corning, and such as alkyl dimethicone copolyols, for example the lauryl methicone copolyol sold under the name “Dow Corning 5200 Formulation Aid” by Dow Corning, the cetyl dimethicone copolyol sold under the name Abil EM 90® by Goldschmidt-Degussa (INCI name: Cetyl PEG/PPG-10/1 dimethicone) and the polyglyceryl-4 isostearate/cetyl dimethicone copolyol/hexyl laurate mixture sold under the name Abil WE 09® by Goldschmidt-Degussa (INCI name: polyglyceryl-4 isostearate/hexyl laurate/cetyl PEG/PPG-10/1 dimethicone). It is also possible to add thereto one or more coemulsifiers which, advantageously, can be chosen from the group consisting of esters of a fatty acid comprising a branched chain and of a polyol and in particular esters of a fatty acid comprising a branched chain and of glycerol and/or of sorbitan, for example polyglyceryl isostearate, such as the product sold under the name Isolan G134® by Goldschmidt-Degussa, sorbitan isostearate, such as the product sold under the name Arlacel 987® by Uniqema, glyceryl sorbitan isostearate, such as the product sold under the name Alacel 986® by Uniqema, and their mixtures. These coemulsifiers can be directly as a mixture with the emulsifier, as is the case for Abil WE 09.

Mention may be made, as emulsifiers which can be used in the preparation of the O/W emulsions, for example, of non-ionic emulsifiers, such as esters of fatty acids and of oxyalkylene (more particularly polyoxyethylene) polyols, for example polyethylene glycol stearates, such as PEG-100 stearate, PEG-50 stearate and PEG-40 stearate; and mixtures comprising them, such as the mixture of glyceryl stearate and polyethylene glycol stearate (100 EO) (INCI name: Glyceryl Stearate/PEG-100 Stearate) sold under the name Simulsol 165® by Seppic or under the name Arlacel 165® by Uniqema; oxyalkylenated esters of fatty acids and of sorbitan comprising, for example, from 20 to 100 EO units, for example those sold under the trade names Tween 20® or Tween 60® by Uniqema; oxyalkylenated (oxyethylenated and/or oxypropylenated) ethers of fatty alcohols; sugar esters, such as sucrose stearate; and their mixtures.

It is possible to add coemulsifiers to these emulsifiers, such as, for example, fatty alcohols having from 8 to 26 carbon atoms, such as cetyl alcohol, stearyl alcohol and their mixture (cetearyl alcohol), octyldodecanol, 2-butyloctanol, 2-hexyldecanol, 2-undecylpentadecanol or oleyl alcohol.

It is also possible to prepare emulsions without emulsifiers or comprising less than 0.5% of the total weight of the composition thereof by using appropriate compounds, for example polymers having emulsifying properties, such as the polymers sold under the names Carbopol 1342 and Pemulen by Noveon, or polymers and copolymers of 2-acrylamido-2-methylpropanesulphonic acid (AMPS). It is also possible to prepare emulsions without emulsifiers stabilized by silicone particles or particles of metal oxide, such as TiO₂, or others.

Adjuvants

The composition used for impregnating the substrate can additionally comprise adjuvants including those conventionally employed in the cosmetics and dermatological field. It can in particular comprise one or more adjuvants chosen from foaming surfactants, organic solvents, softenings, antioxidants, chelating agents, fragrances, UV screening agents, colouring materials, hydrophilic or lipophilic active principles, hydrophilic gelling agents, preservatives, lipid vesicules which can optionally encapsulate one or more active principles, or any other ingredient commonly used in cosmetics or dermatology, and their mixtures. The amounts of these various adjuvants are those conventionally used in the fields under consideration. Of course, these adjuvants must be of such a nature and used in such an amount that they do not interfere with the composition according to the invention. The amount of these adjuvants can range, for example, from 0.01 to 30% by weight, with respect to the total weight of the composition.

Mention may be made, as foaming surfactants which can be present in the composition according to the invention, in particular for articles, especially wipes, for cleaning or removing make-up from the skin, for example, of:

(1) among non-ionic surfactants, oxyethylene/oxypropylene block polymers, such as Poloxamer 184 (INCI name); alkylpolyglycosides and in particular alkylpolyglucosides (APGs) having an alkyl group comprising from 6 to 30 carbon atoms (C₆-C₃₀ alkyl polyglucosides) and preferably 8 to 16 carbon atoms, such as, for example, decyl glucoside (alkyl-C9/C11-polyglucoside(1.4)), such as the product sold under the name Mydol 10 by Kao Chemicals, the product sold under the name Plantaren 2000 UP or Plantacare 2000 UP by Cognis and the product sold under the name Oramix NS 10 by Seppic; caprylyl/capryl glucoside, such as the product sold under the name Oramix CG 110 by Seppic; lauryl glucoside, such as the products sold under the names Plantaren 1200 N and Plantacare 1200 by Cognis; and coco glucoside, such as the product sold under the name Plantacare 818/UP by Cognis;

(2) among anionic surfactants, alkyl sulphates, alkyl ether sulphates and their salts, in particular their sodium salts, such as the sodium laureth sulphate/magnesium laureth sulphate/sodium laureth-8 sulphate/magnesium laureth-8 sulphate mixture sold under the name of Texapon ASV by Cognis; the sodium lauryl ether sulphate (C₁₂₋₁₄ 70/30) (2.2 EO) sold under the names Sipon AOS 225 or Texapon N702 Paste by Cognis; the ammonium lauryl ether sulphate (C₁₂₋₁₄ 70/30) (3 EO) sold under the name Sipon Lea 370 by Cognis; the ammonium (C₁₂-C₁₄)alkyl ether (9 EO) sulphate sold under the name Rhodapex AB/20 by Rhodia Chimie;

(3) among amphoteric or zwitterionic surfactants, alkylamidoalkylamine derivatives, such as the N-disodium N-cocoyl-N-carboxymethoxyethyl-N-(carboxymethyl)ethylenediamine (INCI name: Disodium cocoamphodiacetate) sold in saline aqueous solution under the name Miranol C2M CONC. NP by Rhodia Chimie; N-sodium N-cocoyl-N-hydroxyethyl-N-(carboxymethyl)ethylenediamine (INCI name: sodium cocamphoacetate) and the mixture of coconut acid ethanolamides (INCI name: Cocamide DEA).

Furthermore, the composition added to or impregnated onto the article of the invention can comprise one or more hydrophilic gelling agents. These gelling agents can be chosen in particular from natural polymers or synthetic polymers. Mention may be made, as natural polymers, of polysaccharides, such as gums, in particular xanthan gum, and cellulose derivatives, such as hydroxymethylcellulose and hydroxypropylcellulose. Mention may be made, as synthetic polymers, of modified or unmodified carboxyvinyl polymers, such as the products sold under the names Carbopol (INCI name: carbomer) and Pemulen (INCI name: Acrylates/C10-30 alkyl acrylate crosspolymer) by Noveon; or such as the crosslinked sodium polyacrylate sold under the name Cosmedia SP by Cognis (INCI name: Sodium polyacrylate) or Norsocryl S35, sold by Atofina; or polyacrylates and polymethyacrylates, such as the products sold under the names Lubrajel and Norgel by Guardian or under the name Hispagel by Hispano Chimica; polyacrylamides; optionally crosslinked and/or neutralized polymers and copolymers of 2-acrylamido-2-methylpropanesulphonic acid, such as the poly(2-acrylamido-2-methylpropanesulphonic acid) sold by Clariant under the name Hostacerin AMPS (INCI name: ammonium polyacryldimethyltauramide); crosslinked anionic copolymers of acrylamide and of AMPS which are provided in the form of a W/O emulsion, such as those sold under the name Sepigel 305 (INCI name: Polyacrylamide/C13-14 Isoparaffin/laureth-7) and Simulgel 600 (INCI name: Acrylamide/Sodium acryloyldimethyltaurate copolymer/isohexadecane/polysorbate 80) by Seppic. The amount of hydrophilic gelling agent(s) must be such that it does not interfere with the qualities of the composition, that is to say that the composition can be spread over the support to be impregnated. This amount can range, for example, from 0.01 to 5% by weight, preferably from 0.05 to 3% by weight and better still from 0.05 to 1% by weight, with respect to the total weight of the composition.

The composition according to the invention can additionally comprise one or more fillers. The composition can comprise, as fillers, for example, inorganic particles, such as clays, silicas, metal oxides, such as titanium dioxide or zinc oxide, or mica, and/or organic fillers, such as polyamide (Nylon) particles and in particular those sold under the names Orgasol by Atochem; polyethylene powders; microspheres based on acrylic copolymers, such as those made of ethylene glycol dimethacrylate/lauryl methacrylate copolymer sold by Dow Corning under the name Polytrap; polymethyl methacrylate microspheres sold under the name Microsphere M-100 by Matsumoto or under the name Covabead LH85 by Wackherr; ethylene/acrylate copolymer powders, such as those sold under the name Flobeads by Sumitomo Seika Chemicals; expanded powders, such as hollow microspheres and in particular the microspheres formed from a terpolymer of vinylidene chloride, acrylonitrile and methacrylate and sold under the name Expancel by Kemanord Plast; powders formed from natural organic materials, such as starch powders, in particular powders formed from crosslinked or non-crosslinked maize, wheat or rice starches, such as powders formed from starch crosslinked with octenyl succinic anhydride sold under the name Dry-Flo by National Starch; silicone resin microbeads, such as those sold under the name Tospearl by Toshiba Silicone, in particular Tospearl 240; and their mixtures. The amount of filler(s) can range, for example, from 0.05 to 10% by weight and better still from 0.1 to 5% by weight, with respect to the total weight of the composition.

Mention may be made, as active principles which can be used in the compositions of the invention, for example, of moisturizing agents, such as protein hydrolysates; sodium hyaluronate; polyols, such as glycerol, glycols, such as polyethylene glycols, and sugar derivatives; anti-inflammatory; procyanidol oligomers; vitamins, such as vitamin A (retinol), vitamin E (tocopherol), vitamin C (ascorbic acid), vitamin B5 (panthenol), vitamin B3 (niacinamide), the derivatives of these vitamins (in particular esters) and their mixtures; urea; caffeine; depigmenting agents, such as kojic acid, hydroquinone and caffeic acid; salicylic acid and its derivatives; α-hydroxy acids, such as lactic acid and glycolic acid and their derivatives; retinoids, such as carotenoids and vitamin A derivatives; sunscreens, algae, fungal, plant, yeast or bacterial extracts; antiseborrhoeic active principles which make it possible to clean from excess sebum on the skin; antibacterial active principles, such as 2,4,4′-trichloro-2′-hydroxydiphenyl ether (or triclosan), 3,4,4′-trichlorocarbanilide (or trichlocarban) and the acids indicated above, in particular salicylic acid and its derivatives; mattifying agents; tightening agents; ceramides; essential oils; and their mixtures; and any active principle appropriate for the final objective of the composition.

The active principle or principles can, for example, be present in a concentration ranging from 0.01 to 20%, preferably from 0.1 to 5% and better still from 0.5 to 3% of the total weight of the composition.

Of course, a person skilled in the art will take care to choose the optional compound or compounds to be added to the compositions of the invention so that the advantageous properties intrinsically attached to the product in accordance with the invention are not, or not substantially, detrimentally affected by the envisaged addition.

Substrate

The water-insoluble substrate can comprise one or more layers and it can be chosen from the group consisting of woven materials, non-woven materials, foams, sponges, waddings, as sheets, balls or films, etc. It can in particular be a non-woven substrate based on fibres of natural origin (flax, wool, cotton, silk, fibres made of bamboo) or synthetic origin (cellulose derivatives, viscose, polyvinyl derivatives, polyesters, such as poly(ethylene terephthalate), polyolefins, such as polyethylene (PET) or polypropylene, polyamides, such as Nylon, or acrylic derivatives) and their mixtures, such as viscose/PET, polylactic acid (PLA) or viscose/polylactic acid (viscose/PLA). The substrate is preferably selected from woven materials, non-woven materials, foams, sponges waddings, and combinations thereof.

According to a preferred embodiment of the invention, the substrate is a non-woven material. A general description of non-woven materials is given in Riedel “Non-woven Bonding Methods and Materials”, Non-woven World (1987). These substrates are according to the normal methods of the technology for the preparation of non-woven materials.

When the substrate is a non-woven material, use is preferably made of a non-woven material which does not go into ball and which is sturdy enough not to disintegrate and not to become fluffy when applied to the skin. It must be absorbent and soft at least on one face for the removal of make-up from the eyes in particular. Mention may be made, as appropriate non-woven materials, for example, of those sold under the names Ultraloft 15285-01, Ultraloft 182-008, Ultraloft 182-010 and Ultraloft 182-016 by BBA, Vilmed M1519 Blau, Vilmed M 1550 N and 112-132-3 by Freudenberg, that sold under the name Norafin 11601-010B by Jacob Holm Industries, the flocked non-woven materials sold under the names Univel 109 and Univel 119 by Uni Flockage and that made of viscose/PLA supplied by Sandler.

This substrate can comprise one or more layers having identical or different properties and having properties of elasticity and of softness and other properties appropriate to the desired use. The substrates can comprise, for example, two parts having different elasticity properties, as described in the document WO-A-99/13861, or can comprise a single layer having different densities, as described in the document WO-A-99/25318, or can comprise two layers of different textures, as described in the document WO-A-98/18441.

In addition, when the article is used for the body, the substrate can comprise at least one rough face for making it possible, at the same time, to massage the skin or to scrub the skin.

As indicated above, the substrate can have any size and any shape which are appropriate for the desired objective. Furthermore, it generally has a surface area of between 0.005 m² and 0.1 m², preferably 0.01 m² and 0.05 m².

The degree of composition addition to or impregnation onto/into the substrate generally ranges from 100 to 1000%, preferably from 250 to 700%, of the weight of the substrate. The techniques for adding to/impregnating the substrates with compositions are well known in this field and can all be applied in the present invention. Generally, the composition is heated and added to the substrate by one or more techniques comprising immersion, coating, vaporization, spraying, pouring, and the like.

Another subject-matter of the invention is a method for caring for the skin or cleaning and/or removing make-up from the skin which consists in applying, to the skin, an article as defined above.

### COMPOSITION EXAMPLES

The examples below of compositions according to the invention are given by way of illustration and without the limiting nature. The names are as chemical name or as INCI name. The amounts are given therein as % by weight, unless otherwise mentioned.

### Example 1

### W/O Emulsion

| | |
|---|---|
| Compounds | Amounts in % |

Phase A

| | |
|---|---|
| Cetyl PEG/PPG-10/1 dimethicone (Abil EM90) | 1.5 |
| Polyglyceryl-4 isostearate (Isolan GI34) | 0.5 |
| Isohexadecane | 11 |
| Preservatives | 0.1 |
| Poly C10-30 alkyl acrylate (Intelimer IPA- | 1.3 |
| 13.1) | |
| Apricot oil (Prunus armeniaca Oil) | 5 |

Phase B

| | |
|---|---|
| Cyclohexasiloxane | 9 |
| Acrylate copolymer (Cosmedia SP) | 0.5 |
| Aluminium starch octenylsuccinate (Dry-Flo) | 1.5 |

Phase C

| | |
|---|---|
| Water | 58.4 |
| Preservatives | 0.5 |
| Glycerol | 10 |
| Magnesium sulphate | 0.7 |

Procedure:

Phase A was heated at 70° C. with stirring until a homogeneous phase is obtained and then brought back to 65° C.

Phase B was stirred at ambient temperature until a satisfactory dispersion of the fillers is obtained and was then added to phase A.

Phase C was subsequently heated with stirring at 85° C. until a clear phase is obtained and then the temperature was brought back to 65° C.

Phase C was subsequently added to the mixture A+B for emulsification using a Moritz stirrer. The mixture was brought back to 25° C.

A viscous W/O emulsion was obtained which is capable of homogeneously impregnating a substrate in the form of a wipe. For the impregnation of the substrate, the emulsion was reheated to 60° C., which rendered it fluid, and then it was dispersed over the substrate under hot conditions. The substrate was a non-woven material of viscose/PLA type in the form of a wipe, the degree of impregnation being 350%. The impregnated wipe was subsequently cooled to ambient temperature (approximately 20° C.).

The impregnated wipe thus obtained can be used for caring for the skin. It can also be applied as a mask to the face.

Procedure:

Phase A was heated at 70° C. with stirring until a homogeneous phase is obtained and then brought back to 65° C.

Phase B was stirred at ambient temperature until a satisfactory dispersion of the fillers is obtained and was then added to phase A.

Phase C was subsequently heated with stirring at 85° C. until a clear phase is obtained and then the temperature was brought back to 65° C.

Phase C was subsequently added to the mixture A+B for emulsification using a Moritz stirrer. The mixture was brought back to 25° C.

A viscous W/O emulsion was obtained which is capable of homogeneously impregnating a substrate in the form of a wipe. For the impregnation of the substrate, the emulsion was reheated to 60° C., which rendered it fluid, and then it was dispersed over the substrate under hot conditions. The substrate was a non-woven material of viscose/PLA type in the form of a wipe, the degree of impregnation being 350%. The impregnated wipe was subsequently cooled to ambient temperature (approximately 20° C.).

### Example 2

### W/O Emulsions

| | | |
|---|---|---|
| | Composition | |
| | according to | |
| | the | Comparative |
| | invention | Composition |
| Compounds | (%) | (%) |

Phase A

| | | |
|---|---|---|
| Cetyl PEG/PPG-10/1 dimethicone | 1.5 | 1.5 |
| (Abil EM90) | | |
| Polyglyceryl-4 isostearate (Isolan | 0.5 | 0.5 |
| GI34) | | |
| Isohexadecane | 11 | 11 |
| Preservatives | 0.1 | 0.1 |
| Poly C10-30 alkyl acrylate | 1 | — |
| (Intelimer IPA-13.1) | | |
| Dextrine palmitate (Rheopearl Tl, | — | 1 |
| Chiba Flour) | | |
| Apricot oil (Prunus armeniaca Oil) | 5 | 5 |

Phase B

| | | |
|---|---|---|
| Cyclohexasiloxane | 9 | 9 |
| Acrylate copolymer (Cosmedia SP) | 0.5 | 0.5 |
| Aluminium starch octenylsuccinate | 1.5 | 1.5 |
| (Dry-Flo) | | |

Phase C

| | | |
|---|---|---|
| Water | 58.4 | 58.4 |
| Preservatives | 0.5 | 0.5 |
| Glycerol | 10 | 10 |
| Magnesium sulphate | 0.7 | 0.7 |

Procedure:

Phase A was heated at 70° C. with stirring until a homogeneous phase is obtained and then brought back to 40° C.

Phase B was stirred at ambient temperature until a satisfactory dispersion of the fillers is obtained and was then added to phase A.

Phase C was subsequently heated with stirring at 85° C. until a clear phase is obtained and then the temperature was brought back to 25° C.

Phase C was subsequently added to the mixture A+B for emulsification using a Moritz stirrer.

Viscous W/O emulsions were obtained.

For the impregnation of the substrate, each emulsion was reheated to 65° C., and then it was dispersed over the substrate under hot conditions. The substrate was a non-woven material of viscose/PLA type in the form of a wipe, the degree of impregnation being 350%. The impregnated wipe was subsequently cooled to ambient temperature (approximately 20° C.).

The composition according to the invention when reheated to 65° C., becomes fluid and homogeneously impregnates the substrate in the form of a wipe. The comparative composition, when reheated to 65° C., stays very viscous and does not become fluid: it is not possible to impregnate homogeneously the substrate with the composition.

The use of a semi-crystalline polymer makes it possible to obtain a good and homogeneous impregnation of the composition onto the substrate.

The above written description of the invention provides a manner and process of making and using it such that any person skilled in this art is enabled to make and use the same, this enablement being provided in particular for the subject matter of the appended claims, including an article comprising (A) a water-insoluble substrate, comprising one or more layers, and (B) a composition added to or impregnated onto the substrate, comprising at least one aqueous phase, at least one oily phase, and one or more semi-crystalline polymers.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” etc. preface examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. An article comprising (A) a water-insoluble substrate, comprising one or more layers, and (B) a composition added to or impregnated onto the substrate, comprising at least one aqueous phase, at least one oily phase, and one or more semi-crystalline polymers.

2. The article according to claim 1, wherein the one or more semi-crystalline polymers are chosen from homopolymers and copolymers resulting from the polymerization of at least one monomer comprising crystallizable chain(s), the crystallizable chain being chosen from alkyl chains comprising at least 11 carbon atoms and at most 40 carbon atoms.

3. The article according to claim 1, wherein the one or more semi-crystalline polymers are chosen from homopolymers resulting from the polymerization of a monomer comprising a crystallizable chain chosen from saturated C₁₄-C₂₂ alkyl acrylates and saturated C₁₄-C₂₂ alkyl methacrylates and copolymers obtained by copolymerization of a monomer comprising a crystallizable chain chosen from saturated C₁₄-C₂₂ alkyl acrylates and saturated C₁₄-C₂₂ alkyl methacrylates with acrylic acid.

4. The article according to claim 1, comprising at least one semi-crystalline polymer selected from the group consisting of stearyl acrylate homopolymer, behenyl acrylate homopolymer, and their blends.

5. The article according to claim 1, wherein the amount of semi-crystalline polymer(s) ranges from 0.1 to 20% by weight, with respect to the total weight of the composition (B).

6. The article according to claim 1, wherein the amount of oily phase ranges from 2 to 70% by weight with respect to the total weight of the composition (B).

7. The article according to claim 1, wherein the composition (B) further comprises at least one emulsifier.

8. The article according to claim 1, wherein the composition (B) further comprises one or more hydrophilic gelling agents.

9. The article according to claim 8, wherein the amount of hydrophilic gelling agent(s) ranges from 0.01 to 5% by weight, with respect to the total weight of the composition (B).

10. The article according to claim 1, wherein the composition (B) is an emulsion.

11. The article according to claim 1, wherein the composition (B) exhibits a viscosity from 1 to 15 Pa·s.

12. The article according to claim 1, wherein the substrate is selected from the group consisting of woven materials, non-woven materials, foams, sponges waddings, and combinations thereof.

13. The article according to claim 1, wherein the substrate is a non-woven material comprising fibres of natural origin and/or of synthetic origin.

14. The article according to claim 1, wherein it comprises from 100 to 1000% by weight of composition (B), with respect to the weight of substrate (A).

15. The article according to claim 1, wherein it constitutes an article for caring for the skin and/or hair and/or for cleaning and/or removing make-up from the skin.

16. The article according to claim 1, wherein it constitutes a cosmetic article.

17. A method for caring for the skin or hair and/or cleaning and/or removing make-up from and/or scrubbing the skin, comprising contacting the skin or the hair with the article of claim 1.

18. A method for caring for the skin or hair and/or cleaning and/or removing make-up from and/or scrubbing the skin, comprising contacting the skin or the hair with the article of claim 16.
